(19) United States
(12) Patent Application Publication
(10) Pub. No.: US 2018/0036035 A1
(43) Pub. Date: Feb. 8, 2018

(54) Method and System for Connecting a Lead to Cardiac Tissue

(71) Applicants: Demcon Advanced Mechatronics B.V., Enschede (NL); Academisch Ziekenhuis Gronigen, Groningen (NL)

(72) Inventors: Maria Cornelia Ruijter, Enschede (NL); Hernes Jacobs, Enschede (NL); Benno Lansdorp, Enschede (NL); Massimo Mariani, Groningen (NL); Theodorus Johannes Klinkenberg, Groningen (NL)

(21) Appl. No.: 15/550,686

(22) PCT Filed: Feb. 13, 2015
(86) PCT No.: PCT/EP2015/053142

§ 371 Date: Aug. 11, 2017

(51) Int. Cl. A61B 17/34; A61B 18/14; A61N 1/05

(52) U.S. Cl. CPC A61B 17/3468; A61N 1/0587; A61B 17/3421; A61B 18/1482; A61B 2017/3488; A61B 2017/306

(57) ABSTRACT

The invention relates to a method of connecting a lead to cardiac tissue in a patient's body, comprising the steps of: a) providing an elongated hollow delivery tool having a proximal end and a distal end, b) inserting the distal end of the tool into the patient's body, c) navigating the delivery tool until its distal end contacts the cardiac tissue at a contact location, d) determining if the contact location is suitable for connection of the lead, e) if the contact location is deemed suitable, fixedly connecting an electrode of the lead to the cardiac tissue at that location, and f) if the contact location is not deemed suitable, moving the distal end of the delivery tool away from the cardiac tissue and navigating the delivery tool until its distal end contacts the cardiac tissue at an alternative contact location that is spaced apart from the previous contact location, and repeating step d). The invention further relates to a system for connecting a lead to cardiac tissue in a patient's body, comprising: an elongated hollow delivery tool having a proximal end and a distal end, the distal end being arranged for insertion into the patient's body, a navigating device for navigating the distal end of the delivery tool to a contact location on the cardiac tissue, a determinator for determining the suitability of the contact location, and a connecting device for fixedly connecting an electrode of the lead to the cardiac tissue at a selected contact location.

The invention relates to novel ways of introducing a medical lead, in particular a lead for an implantable medical device, into a patient's body and of connecting such lead to body tissue, in particular cardiac tissue.

When a lead for a medical device, like e.g. a pacemaker or defibrillator is connected to cardiac tissue, it is important that the lead is positioned for maximum effectiveness. In particular, the location of the connection of the lead should allow the medical device to support and/or control cardiac functions in an optimum manner.

When a lead is to be connected to an outer surface of a patient's heart (an epicardial lead), this conventionally requires an open chest surgical procedure (thoracotomy). After an incision has been made in the patient's chest and surrounding tissue has been peeled back to give access to the heart, an electrode oat the end of the lead is connected to the patient's heart. This connection is made by implanting the electrode in the cardiac tissue. The location where the electrode is to be implanted is selected on the basis of visual inspection. After connecting the lead to the cardiac tissue, the connection is tested and if this is found to be satisfactory, the patient's chest is closed again.

Such an open chest procedure is complicated and results in severe trauma and scarring of the chest, requiring a relatively long healing period. Moreover, mere visual inspection does not allow determining an optimum location where the lead is to be delivered, since such inspection does not provide a clear indication of the relevant condition of the local cardiac tissue, in particular its electric activity. Thus, there is always a risk that the electrode will be implanted in a part of the cardiac tissue that is not electrically active, e.g. due to previous infarct. In that case, the electrode has to be removed and repositioned to another location, where it has to be implanted again. This means that the patient has to spend more time in surgery, which increases the risk of complications. Moreover, removing and reimplanting the electrode leads to additional trauma (risk of bleeding) and scarring of the cardiac tissue.

It is an object of the invention to provide a method that will allow a medical lead to be connected to cardiac tissue at an optimum location. It is a further object of the invention to provide a method that can be performed using minimally invasive surgery.

It is yet another object of the invention to provide a system with which an operator may accurately position a medical lead and may easily connect the lead to cardiac tissue using laparoscopic techniques.

In one embodiment the invention provides a method of connecting a lead to cardiac tissue in a patient's body, comprising the steps of: a) providing an elongated hollow delivery tool having a proximal end and a distal end; b) inserting the distal end of the tool into the patient's body; c) navigating the delivery tool until its distal end contacts the cardiac tissue at a contact location; d) determining if the contact location is suitable for connection of the lead; e) if the contact location is deemed suitable, fixedly connecting an electrode of the lead to the cardiac tissue at that location; and f) if the contact location is not deemed suitable, moving the distal end of the delivery tool away from the cardiac tissue and navigating the delivery tool until its distal end contacts the cardiac tissue at an alternative contact location that is spaced apart from the previous contact location, and repeating step d).

By navigating the delivery tool it can be moved to a predetermined location on the patient's heart through a small incision. And by determining the suitability of a location after contact has been made, more information about the condition of the cardiac tissue can be obtained than from mere visual observation. Moreover, because the suitability of the location is assessed before the lead is actually implanted in the heart, time-consuming positioning by ‘trial and error’ can be avoided.

In order to allow optimum information transfer from the cardiac tissue to the delivery tool, step d) includes temporarily stabilising the distal end of the delivery tool to the cardiac tissue and performing measurements while the distal end is stabilised.

When the distal end of the delivery tool is temporarily fixed to the cardiac tissue by suction, scarring or bleeding of the tissue is avoided.

This can be done simply and swiftly when a suction cup is deployed from the distal end of the delivery tool, placed on the cardiac tissue and operatively connected to a source of suction.

In order to undo the temporary stabilisation, step f) may then include releasing the suction before moving the distal end of the delivery tool away from the cardiac tissue.

When performing the measurements includes measuring local electrical activity in the cardiac tissue, an accurate assessment of the suitability of the location can be made.

The local electric activity may be measured by the electrode at the tip of the lead to be connected. In this way there is no need for additional sensing equipment to be inserted into the patient's body.

Alternatively or additionally, the local electric activity may be measured by a member used for temporarily stabilising the delivery tool to the cardiac tissue, e.g. by a sensor arranged in the suction cup. Here again, no additional sensing equipment is required.

After the lead has been connected to the cardiac tissue, the method may further comprise the step of: g) retracting the distal end of the delivery tool from the patient's body.

The distal end of the delivery tool may advantageously be inserted into the patient's body by first introducing a hollow insertion tool into the patient's body and then guiding the distal end of the delivery tool through the hollow insertion tool, which may for instance be a trocar. In this way the lead may be delivered to the patient's heart by minimally invasive surgery, thus reducing trauma, scarring and recovery time.

In that case step g) may includes the substeps of: g1) retracting the distal end of the delivery tool through the hollow insertion tool, and g2) retracting the hollow insertion tool from the patient's body.

In order to maintain the connection between the lead and the cardiac tissue and avoid stressing the connection, additional lead material may be guided through the delivery tool while its distal end is retracted from the patient's body.

In another embodiment the invention provides a system for connecting a lead to cardiac tissue in a patient's body, comprising: an elongated hollow delivery tool having a proximal end and a distal end, the distal end being arranged for insertion into the patient's body; a navigating device for navigating the distal end of the delivery tool to a contact location on the cardiac tissue; a determinator for determining the suitability of the contact location; and a connecting device for fixedly connecting an electrode of the lead to the cardiac tissue at a selected contact location. Such a tool allows the lead to be positioned on the heart using minimally invasive surgery and makes it possible to assess the condition of the cardiac tissue and its suitability for electrical connection to the lead in a reliable manner by direct contact.

The system may advantageously comprise a stabilising device for temporarily stabilising the distal end of the delivery tool to the cardiac tissue, and as explained above the stabilising device may be a suction device.

A structurally simple suction device is obtained when the stabilising device includes a suction cup that is deployable from the distal end of the delivery tool and that is connectable to a source of suction.

In order to allow the delivery tool to be easily inserted into and navigated in the patient's body, the system may further comprise a cover member for covering the suction cup, while the cover member and the suction cup may be movable with respect to each other. Covering the suction cup reduces the effective diameter of the delivery tool, thus allowing it to be guided through the hollow insertion tool or trocar.

The determinator may advantageously comprise a measuring device for measuring local electrical activity in the cardiac tissue. As explained above, the measuring device may include the electrode at the tip of the lead to be connected. Alternatively or additionally, the measuring device may include a sensor that is integrated into the stabilising device. In any event, the need for additional sensing equipment is obviated.

When the sensor is connected to the electrode of the lead, sensing may be performed by using the lead to transmit electrical signals, so that there is no need for additional wiring or circuitry.

In order to provide the operator with the required amount of control over the delivery device, the measuring device may include a processing unit arranged outside the patient's body and coupled to the lead and/or sensor through the proximal end of the delivery tool.

For easy manipulation, the proximal end of the delivery tool may be attached to a grip accommodating the navigating and connecting devices.

In order to provide for optimum manoeuvrability, the delivery tool may be rotatably mounted on the grip and the distal end of the delivery tool may be bendable, and the navigating device may be arranged for rotating the delivery tool and/or bending the distal end thereof.

In an ergonomically advantageous embodiment of the system, the connecting device is operable by a trigger that is movably arranged on the grip.

When the system further comprises a suction line connecting the suction cup to the source of suction and closable by a valve that is arranged in the grip, the suction cup can be deployed and activated from the grip.

In accordance with a further embodiment, the invention provides a delivery tool for use in a system as described above.

The invention will now be illustrated by way of an exemplary embodiment thereof, with reference being made to the annexed drawings, in which:

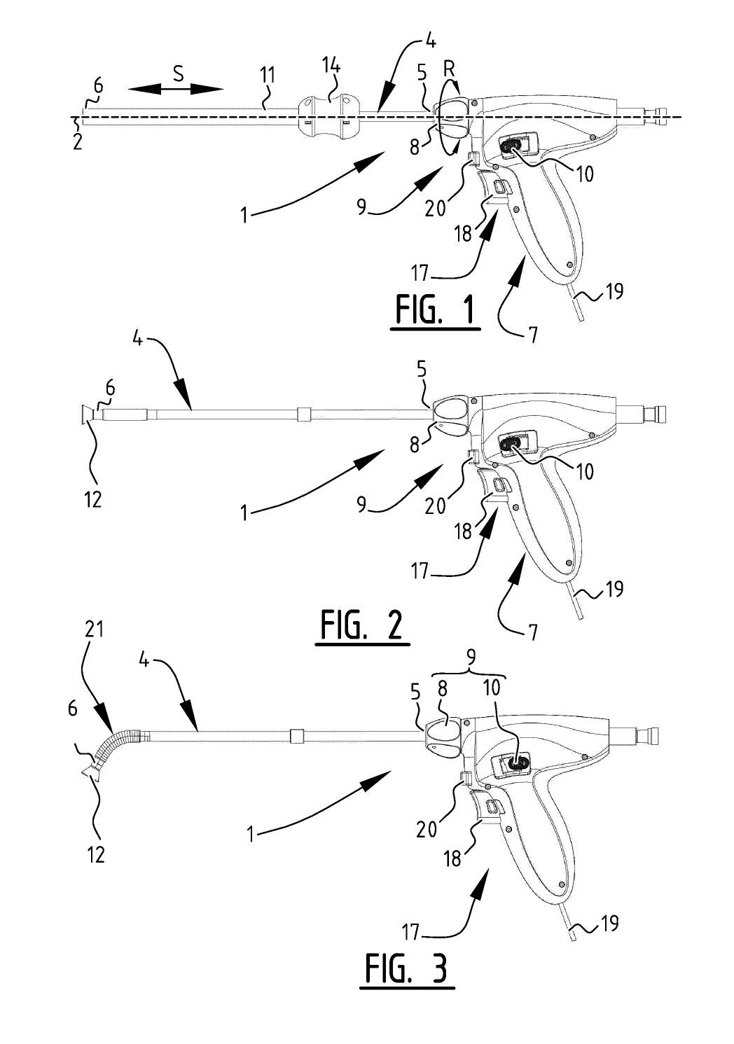

FIG. 1 shows a side view of a delivery tool in accordance with an embodiment of the invention, in which the movable cover is shown in an extended position,

FIG. 2 is a view corresponding with FIG. 1, in which the cover has been removed for reasons of clarity, thus exposing the distal end and suction cup,

FIG. 3 is a view corresponding with FIG. 2, in which the distal end is shown in a bent configuration,

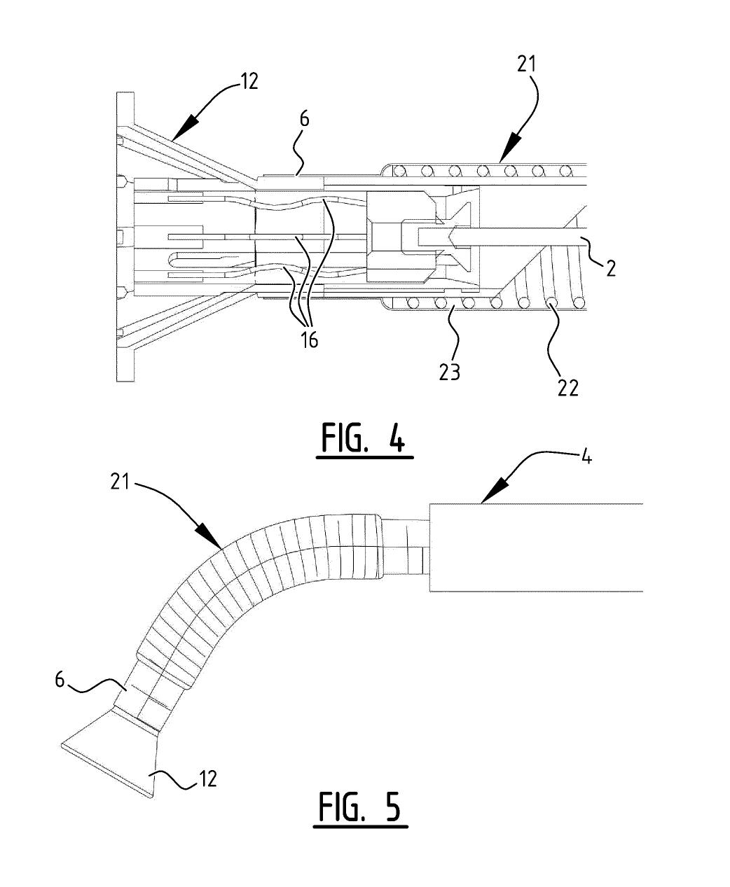

FIG. 4 is a longitudinal sectional view of the distal end of the delivery tool showing the suction cup and a lead having three electrodes at its tip,

FIG. 5 is a detail view of the bent distal end according to arrow V in FIG. 3,

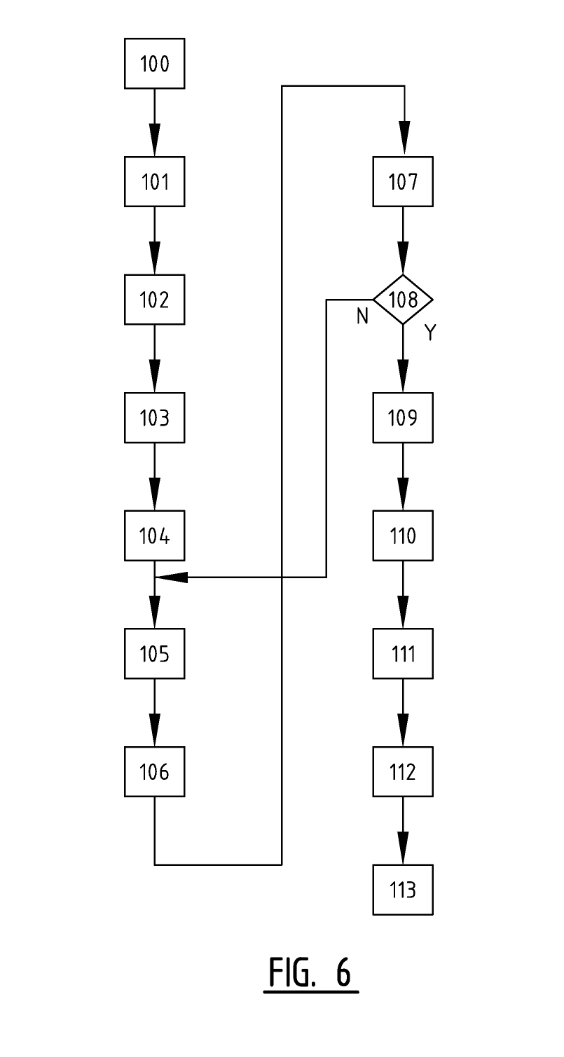

FIG. 6 is a flow diagram showing the various steps of an exemplary method in accordance with an embodiment of the invention,

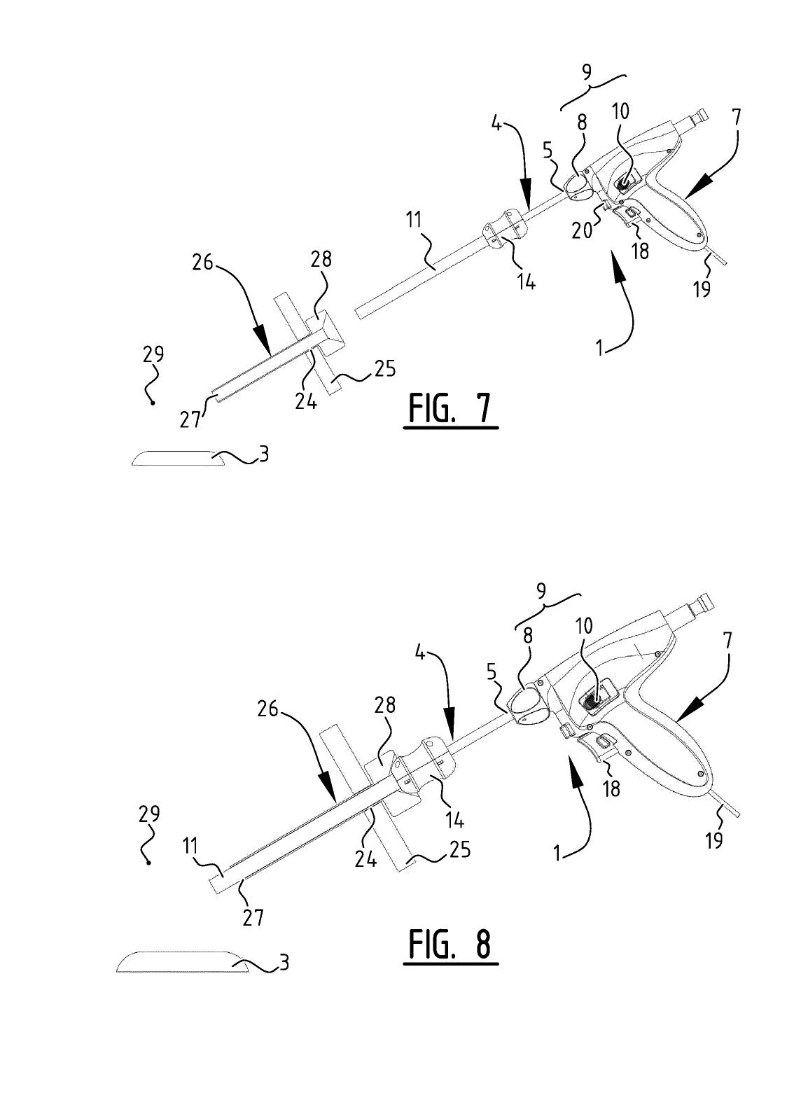

FIGS. 7-10 are side views corresponding with FIGS. 1-3 and illustrating the position and configuration of the delivery tool during four major steps of the method of FIG. 6, and

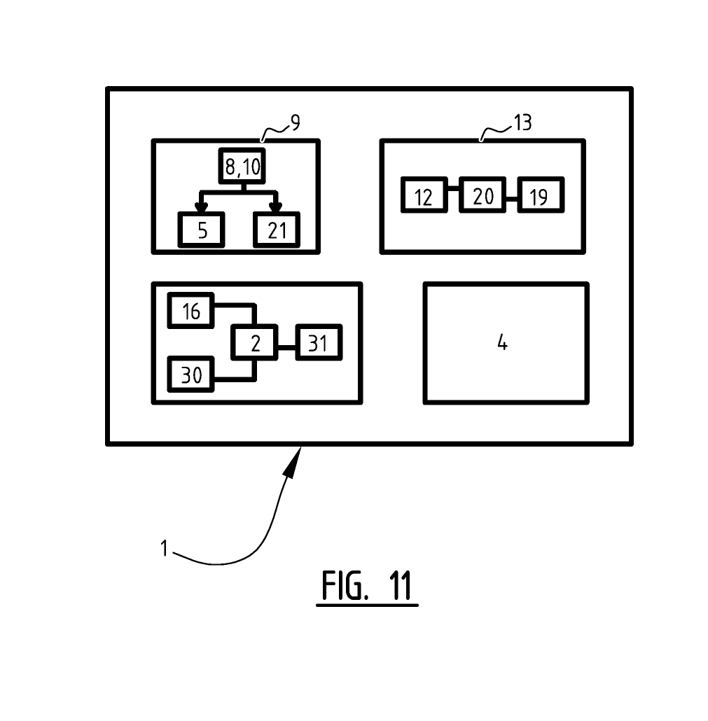

FIG. 11 is a schematic representation of the system and its various components.

A system 1 for connecting a lead 2 (schematically shown as dashed line in FIG. 1) to cardiac tissue 3 in a patient's body comprises an elongated hollow delivery tool 4 having a proximal end 5 and a distal end 6. The proximal end 5 is rotatably mounted on a grip 7 and carries a knob 8 which forms part of a navigating device 9 which will be discussed below. The distal end 6, which in this embodiment is bendable (FIG. 3), is arranged for insertion into the patient's body.

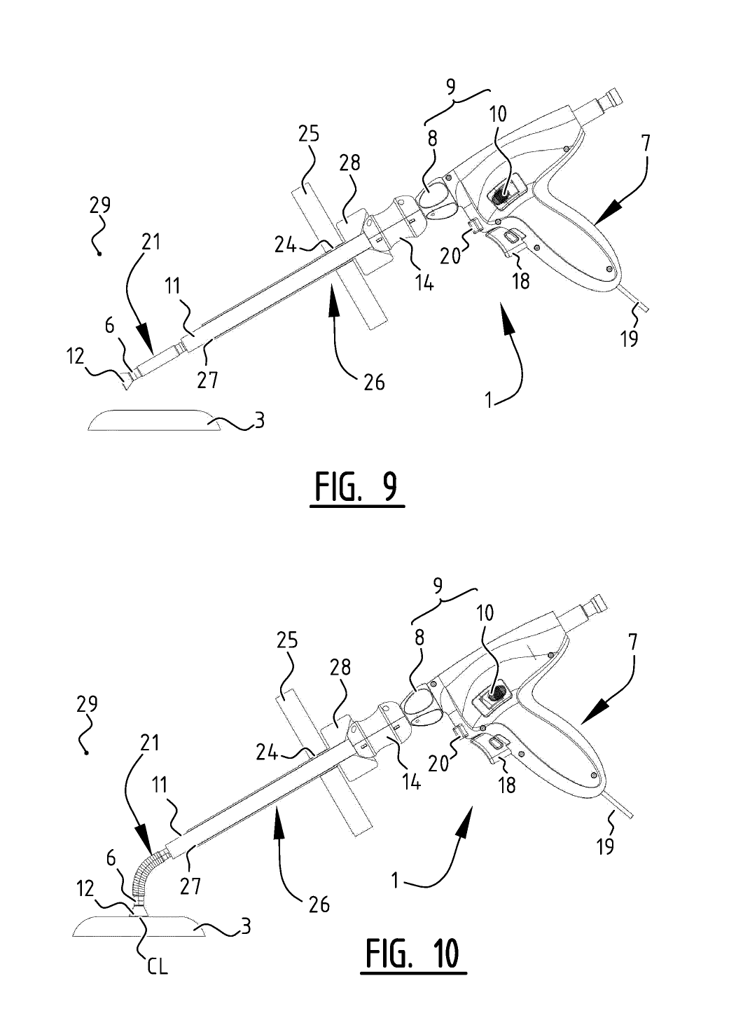

The distal end 6 may be covered by a moveable cover member 11, in this embodiment a slidable sheath, which maintains a suction cup 12 (FIG. 2) on the distal end 6 in a retracted position, thus minimizing the diameter of the distal end 6. This is important since it allows the delivery tool 4 to be inserted into the patient's body using minimally invasive surgery. The suction cup 12 forms part of a stabilising device 13 which will be discussed below. In use the sheath 11 may be slid towards the grip 7 in the direction of the arrow S, thus exposing the distal end 6 and allowing the suction cup 12 to be deployed (FIG. 9). The sheath 11 may be handled by pulling or pushing a knob 14. Alternatively, the sheath 11 may be biased to either its extended or its retracted position.

The system 1 further includes the navigating device 9 mentioned above. This device serves to navigate the distal end 6 of the delivery tool 4 to a contact location CL on the cardiac tissue 3. The navigating device includes the knob 8 for rotating the delivery tool 4 around its longitudinal axis in the direction of arrow R and a slide 10 on the grip 7 for bending the distal end 6. The slide 10 actuates the distal end 6 by means of a wire that extends through the delivery tool 4, and is biased to a position in which the distal end 6 is straight (FIG. 2).

A further component of the system 1 is a determinator 15 (FIG. 11) which is arranged to determine the suitability of the contact location CL on the cardiac tissue 3 for establishing a reliable electrical connection with the lead 2. The determinator 15 includes one or more sensors which may sense the condition of the cardiac tissue locally. These sensors send sensor signals to a processing unit 31, which is arranged outside the patient's body and which is coupled to the sensor (s) by wiring entering the delivery tool 4 through the proximal end 5. The processing unit 31 can transform the sensor signals into an ECG, which can be displayed to an operator.

In the illustrated embodiment the lead 2 itself serves as electrical connection between the sensor(s) and the processing unit 31, so that there is no need for separate dedicated wiring. In this embodiment the sensor(s) are formed by three electrodes 16 protruding from the tip of the lead 2 (FIG. 4). In this way the need for any separate dedicated sensing equipment is obviated. It is also conceivable for the sensor(s) to be formed by another element arranged at or near the distal end 6, like a sensor wire 30 embedded in an edge of the suction cup 12. Here again, use is made of a part of the system that is already present. In order to allow sensor signals to travel over the lead 2, such a sensor wire 30 should then be connected to the lead 2 or the electrodes 16 in some way.

The system 1 also includes a connecting device 17 for fixedly connecting the electrodes 16 of the lead 2 to the cardiac tissue 3 at a selected contact location, which may be the initial contact location CL, but which may also be an alternative contact location at some distance from the initial location, if the initial location is found to be unsuitable, e.g. because of unsatisfactory electrical characteristics. The connecting device 17 includes a trigger 18 that is movably arranged on the grip 7. When depressed, the trigger 18 causes the electrodes 16 to be fixedly connected to the cardiac tissue 3 in any suitable manner.

As stated above, the system further comprises a stabilising device 13 (FIG. 11) for temporarily stabilising the distal end 6 of the delivery tool 4 on the cardiac tissue 3 while its suitability for connecting the lead 2 is being determined. In the illustrated embodiment the stabilising device 13 is a suction device and includes the suction cup 12 that is deployable from the distal end 6 by retracting the slidable sheath 11. The suction cup 12 can be connected to a source of suction by means of a suction line 19, which can run through the hollow delivery tool 4 and then branch off into the grip 7. A valve (not shown here) can be arranged in the grip 7 and can be operable by a switch 20 so as to establish or cease suction at the cup 12.

Bending of the distal end 6 is made possible by a flexible segment 21, which includes a helical spring 22 covered by a flexible skin 23 (FIG. 4). This arrangement allows bend angles of more than 45 degrees to be achieved (FIG. 5). The suction cup 12, which in this embodiment is conical, is also flexible in itself, so that it may be folded together when the sheath 11 is slid over the distal end 6. It may be made of a rubber or elastomer, which must be biocompatible, like all materials used in the delivery tool 4.

An exemplary embodiment of the inventive method of connecting the lead 2 to the cardiac tissue 3 at a suitable contact location CL starts with the provision of a delivery tool 4 (FIG. 6, step 100). Next, a small incision 24 (FIG. 7) is made in the patient's chest 25 (step 101). Then a hollow insertion tool 26, e.g. a trocar is arranged in the incision 24 (step 102).

Subsequently, the distal end 6 of the delivery tool 4, which is covered by the sheath 11, is inserted through the lumen 27 of the insertion tool 26 (FIG. 8 and step 103). In a next step 104 the sheath 11 is retracted, thus freeing the distal end 6 (FIG. 9). In the illustrated embodiment this is done automatically, by contact between the knob 14 and a flange 28 of the insertion tool 26.

Then the distal end 6 is navigated through the patient's thoracic cavity 29 in order to position the distal end at or near the patient's heart (step 105). Such navigating may be done by varying the depth to which the delivery tool 4 is inserted into the thoracic cavity 29, by bending the distal end 6 to a desired degree (slide 10) and by rotating the delivery tool 4 (knob 8).

After the distal end 6 has landed on the cardiac tissue 3 at a contact location CL, it is temporarily stabilized by applying suction to part of the surface of the cardiac tissue 3 (step 106). To this end the suction cup 12 is brought into fluid communication with the source of suction by operating the switch 20 to open the valve in suction line 19. While the distal end 6 is stabilized, measurements of the electrical activity can be performed by the sensor(s) (step 107). Sensor signals can be processed outside the patient's body to generate an ECG, which allows the operator to determine (in step 108) whether or not the initial contact location CL is a suitable location for forming a permanent electrical connection with the lead 2.

If the ECG shows the contact location to be suitable, the connecting means 17 will be actuated by the trigger 18. This will cause the electrode(s) 16 to be moved further forward, protruding beyond the suction cup 12, in order to be fixedly connected to the cardiac tissue 3 (step 109). In principle this connection is intended to be permanent.

If, on the other hand, it is determined that the initial contact location CL is unsuitable, the suction will be interrupted and the distal end 6 will be moved away from the cardiac tissue 3. The method will return to step 105, thus starting a new cycle of navigating, stabilizing, measuring and determining.

After the electrode(s) 16 of the lead 2 have been fixedly connected to the cardiac tissue 3, suction will be interrupted and the distal end 6 will be moved away from the cardiac tissue 3. The distal end 6 will then be straightened so as to allow the sheath 11 to cover the suction cup 12 and allow the delivery tool 4 to be retracted from the patient's thoracic cavity 29 through the insertion tool 26 (step 110). While the delivery tool 4 is distanced from the heart, additional lead material must be paid out sufficiently swiftly to avoid inadvertently pulling the electrodes from the heart again.

Subsequently, the insertion tool (trocar) 26 may be retracted (step 111). Then the lead 2 will be made to measure and will be connected to a medical device that is or has been implanted into the patient's body (step 112), and finally the incision may be closed (step 113).

In this way the method and system of the invention allow an epicardial lead to be connected to cardiac tissue using minimally invasive surgery. Moreover, an optimum location for the lead may be determined before actually establishing a permanent connection.

It should be noted that even in the unlikely event that the connection were to be deemed unsatisfactory, the system also allows a lead to be retracted from the cardiac tissue using minimally invasive surgery. If it were to be determined that for some reason it would be preferable for the lead to be disconnected from the heart, e.g. because the lead and/or the electrode was or became faulty, the delivery tool could be loaded with a pull wire. A forward free end of the pull wire could then be connected to an end of the lead. Then the delivery tool could be inserted through the insertion tool and navigated to the location where the electrode would be connected to the cardiac tissue. The distal end could then again be stabilized on the cardiac tissue before pulling the wire and thus retracting the electrode from the cardiac tissue. Then the delivery tool could be straightened and retracted from the patient's body, taking the electrode with it. However, it is not anticipated that this additional functionality will have to be used often.

Although the invention has been described by way of an exemplary embodiment, it will be clear that many modifications are conceivable. For instance, stabilizing the distal end could be done by other means than suction. Or the determinator could employ other sensors than shown and described here. Other variations, e.g. to the navigating means or the connecting means will occur to the skilled person. Therefore, the scope of the invention is determined solely by the following claims.

1. A method of connecting a lead to cardiac tissue in a patient's body, comprising the steps of:
a) providing an elongated hollow delivery tool having a proximal end and a distal end,
b) inserting the distal end of the tool into the patient's body,
c) navigating the delivery tool until its distal end contacts the cardiac tissue at a contact location,
d) determining if the contact location is suitable for connection of the lead,
e) if the contact location is deemed suitable, fixedly connecting an electrode of the lead to the cardiac tissue at that location, and
f) if the contact location is not deemed suitable, moving the distal end of the delivery tool away from the cardiac tissue and navigating the delivery tool until its distal end contacts the cardiac tissue at an alternative contact location that is spaced apart from the previous contact location, and repeating step d).

2. The method of claim 1, wherein step d) includes temporarily stabilising the distal end of the delivery tool to the cardiac tissue and performing measurements while the distal end is stabilised.

3. The method of claim 2, wherein the distal end of the delivery tool is temporarily stabilised on the cardiac tissue by suction.

4. The method of claim 3, wherein a suction cup is deployed from the distal end of the delivery tool, placed on the cardiac tissue and operatively connected to a source of suction.

5. The method of claim 3, wherein step f) includes releasing the suction before moving the distal end of the delivery tool away from the cardiac tissue.

6. The method of claim 2, wherein performing the measurements includes measuring local electrical activity in the cardiac tissue.

7. The method of claim 6, wherein the local electric activity is measured by the electrode of the lead that is to be connected to the cardiac tissue.

8. The method of claim 6, wherein the local electric activity is measured by a member used for temporarily stabilising the delivery tool on the cardiac tissue.

9. The method of claim 1, further comprising the step of:
g) retracting the distal end of the delivery tool from the patient's body.

10. The method of claim 1, wherein the distal end of the delivery tool is inserted into the patient's body in minimally invasive manner by first introducing a hollow insertion tool into the patient's body and then guiding the distal end of the delivery tool through the hollow insertion tool.

11. The method of claim 9, wherein step g) includes the substeps of:
g1) retracting the distal end of the delivery tool through the hollow insertion tool, and
g2) retracting the hollow insertion tool from the patient's body.

12. The method of claim 9, further comprising guiding additional lead material through the delivery tool while its distal end is being retracted from the patient's body.

13. A system for connecting a lead to cardiac tissue in a patient's body, comprising:
an elongated hollow delivery tool having a proximal end and a distal end, the distal end being arranged for insertion into the patient's body,
a navigating device for navigating the distal end of the delivery tool to a contact location on the cardiac tissue,
a determinator for determining the suitability of the contact location, and
a connecting device for fixedly connecting an electrode of the lead to the cardiac tissue at a selected contact location.

14. The system of claim 13, further comprising a stabilising device for temporarily stabilising the distal end of the delivery tool on the cardiac tissue.

15. The system of claim 14, wherein the stabilising device is a suction device.

16. The system of claim 15, wherein the stabilising device includes a suction cup that is deployable from the distal end of the delivery tool and that is connectable to a source of suction.

17. The system of claim 16, further comprising a cover member for covering the suction cup, wherein the cover member and the suction cup are movable with respect to each other.

18. The system of claim 13, wherein the determinator comprises a measuring device for measuring local electrical activity in the cardiac tissue.

19. The system of claim 18, wherein the measuring device includes the electrode of the lead to be connected.

20. The system of claim 18, wherein the measuring device includes a sensor that is integrated into the stabilising device.

21. The system of claim 20, wherein the sensor is connected to the electrode of the lead.

22. The system of claim 19, wherein the measuring device includes a processing unit arranged outside the patient's body and coupled to the lead and/or sensor through the proximal end of the delivery tool.

23. The system of claim 13, wherein the proximal end of the delivery tool is attached to a grip accommodating the navigating and connecting devices.

24. The system of claim 23, wherein the delivery tool is rotatably mounted on the grip and the distal end of the delivery tool is bendable, and wherein the navigating device is arranged for rotating the delivery tool and/or bending the distal end thereof.

25. The system of claim 23, wherein the connecting device is operable by a trigger that is movably arranged on the grip.

26. The system of claim 23, further comprising a suction line connecting the suction cup to the source of suction and closable by a valve that is arranged in the grip.

27. Delivery tool for use in a system according to claim 13.
